(19) United States
(12) Patent Application Publication
(10) Pub. No.: US 2007/0021835 A1
(43) Pub. Date: Jan. 25, 2007

(54) Systems and methods for providing prostheses

(76) Inventor: Avram Allen Edidin, Portolla Valley, CA (US)

Correspondence Address: KILPATRICK STOCKTON LLP, 1001 WEST FOURTH STREET, WINSTON-SALEM, NC, 27101, US

(21) Appl. No.: 11/485,244

(22) Filed: Jul. 11, 2006

Related U.S. Application Data
(60) Provisional application No. 60/698,290, filed on Jul. 11, 2005.

(51) Int. Cl. A61F 2/44; A61F 2/04; A61F 2/46

(52) U.S. Cl. 623/17.12; 623/17.16; 623/902; 623/23.67

(57) ABSTRACT

Systems and methods for providing prostheses are described. One method comprises identifying a tissue in a treatment area. The method further comprises modifying a structural stiffness of a portion of an expansible body prosthesis disposed adjacent the tissue. The expansible body prosthesis is inserted into the treatment area.

### CROSS-REFERENCE TO RELATED APPLICATIONS

The present application for patent claims priority to U.S. Provisional Patent Application Ser. No. 60/698,290, filed Jul. 11, 2005 and entitled “Systems and Methods for Providing Prostheses,” the disclosure of which is hereby incorporated in full by reference.

### FIELD OF THE INVENTION

The invention relates to systems and methods for providing prostheses for diagnostic or therapeutic purposes.

### BACKGROUND

Intervertebral discs comprise highly specialized joints between adjacent vertebrae from the second cervical vertebra to the first sacral vertebra in the human body. Each intervertebral disc comprises of a strong outer ring called the annulus, and a central nucleus. The annulus is strongly attached to the adjacent vertebral bodies above and below through collagen fibers. The nucleus comprises a mesh of collagen fibers to which is attached proteoglycan molecules that are hygroscopic. The nucleus comprises a central portion and an intermediate zone. The central portion comprises approximately 90% proteoglycan and approximately 10% collagen, and the intermediate zone comprises less proteoglycan and proportionately more collagen. The annulus comprises approximately 90% collagen with about 10% proteoglycan. The proteoglycan may act to allow a sliding motion between adjacent tissue layers during a movement of the human body.

Like many other joints in the body, intervertebral discs, particularly lumbar intervertebral discs, may be subject to various types of injury, degeneration and disease. Painful disc syndromes can develop due to the destruction of the intervertebral disc structure. Back pain emanating from an intervertebral disc may arise from a damaged annulus because the outermost third of the annulus itself is the only part of the disc structure that is innervated.

Accordingly, intervertebral disc herniations are a major source of back pain. Herniations and ruptures of intervertebral discs may also cause pain and numbness in the leg, feet and arms of affected patients. Herniated, or ruptured, discs may be caused by traumatic injury due to accident, illness, the aging process as well as a multiplicity of undefined causes. A herniation of an intervertebral disc may result from a weakened, torn or stretched area of the annulus. Pulp from the nucleus may extrude through the herniated area in the annulus producing pressure on the spinal column or adjacent nerves, thereby causing pain or numbness. Removing the herniated pulp may reduce pressure on the spinal column or adjacent nerves caused by the herniation.

### SUMMARY

Embodiments of the present invention include systems and methods for providing prostheses. One illustrative embodiment comprises an expansible body prosthesis configured to be deployed in a treatment area. The expansible body prosthesis may comprise an aperture configured to receive a filler material. The expansible body prosthesis may comprise a first substantially impermeable portion and a second substantially impermeable portion contiguous to the first substantially impermeable portion. The first substantially impermeable portion has a first structural stiffness, and the second substantially impermeable portion has a second structural stiffness greater than the first structural stiffness. The second substantially impermeable portion may be configured to be disposed adjacent a tissue in the treatment area at least when the expansible body prosthesis is in an expanded state.

This embodiment is mentioned not to limit or define the invention, but to provide an example of an embodiment of the invention to aid understanding thereof. Illustrative embodiments are discussed in the Detailed Description, and further description of the invention is provided there. Advantages offered by the various embodiments of the present invention may be further understood by examining this specification.

### BRIEF DESCRIPTION OF THE FIGURES

These and other features, aspects, and advantages of the present invention are better understood when the following Detailed Description is read with reference to the accompanying drawings, wherein:

FIG. 1 is cross-sectional elevation view of a prosthesis according to one embodiment of the present invention, shown in an expanded state and showing an integral, internal one-way fill valve;

FIG. 2 is a cross-sectional plan view of a prosthesis according to one embodiment of the present invention, shown in an expanded state, and showing a threaded fitting and a conical nose one-way valve coupled to an aperture in the prosthesis, and showing a plurality of strengthened portions of the prosthesis;

FIG. 3 is an assembled elevation view of a delivery system for placement of a prosthesis according to one embodiment of the invention shown in relation to the prosthesis;

FIG. 4 is a perspective view of a human intervertebral disc, wherein a cavity formerly occupied by the nucleus thereof is being accessed by a system configured to insert a prosthesis comprising a strengthened portion according to one embodiment of the present invention prior to deployment of the prosthesis in the nuclear cavity;

FIG. 5 is a perspective view of the assembly of FIG. 4 after deployment of the prosthesis in the nuclear cavity, and prior to retraction of the threaded and barbed coupling thereof into the annulus;

FIG. 6 is a perspective view of the assembly of FIG. 4 after retraction of the threaded and barbed coupling into the annulus, and plugging the annular opening in the intervertebral disc;

FIG. 7 is a perspective view of the assembly of FIG. 4 after the prosthesis has been expanded in the nuclear cavity by provision of a biocompatible filler material therein;

FIG. 8 is a flow chart of a method according to one embodiment of the present invention;

FIG. 9 is a plan view of a sterile kit configured to store a single use tool according to one embodiment of the present invention;

FIG. 10 is an exploded perspective view of the sterile kit of FIG. 9; and

FIG. 11 is a flow chart of a method according to one embodiment of the present invention.

### DETAILED DESCRIPTION

Embodiments of the present invention include systems and methods for providing prostheses. The systems and methods embodying the invention can be adapted for use in many suitable interior body regions, wherever the temporary or permanent replacement of a natural body structure may be required for a therapeutic or diagnostic purpose. The illustrative embodiments show the invention in association with systems and methods used to replace at least a portion of an intervertebral disc. In other embodiments, the present invention may be used in other interior body regions or types of tissues, for example in a human knee joint.

As used in this specification and the appended claims, the singular forms “a,” “an,” and “the” include plural referents unless the context clearly dictates otherwise. Thus, for example, the term “a lumen” is intended to mean a single lumen or a combination of lumens.

Furthermore, the words “proximal” and “distal” refer to directions closer to and away from, respectively, an operator (e.g., surgeon, physician, nurse, technician, etc.). An operator may insert a medical device into a patient, with at least a tip-end (i.e., distal end) of the device inserted inside a patient's body. Thus, in one example, the end of the medical device inserted inside the patient's body would be the distal end of the medical device, while the end of the medical device outside the patient's body would be the proximal end of the medical device. In another example, the entire medical device may be inserted inside the patient's body, where the distal end of the medical device may extend further inside the patient's body than the proximal end of the medical device.

Some embodiments of the present invention comprise a prosthesis configured to replace at least a portion of a human intervertebral disc. For example, one such embodiment comprises an expansible body prosthesis shaped to form a replacement for at least a portion of a nucleus pulposis that has been removed from an intervertebral disc. The expansible body may comprise a body material, for example polyurethane, silicone, Kevlar, polypropylene, other suitable polymers, metals, advanced ceramics, pyrolytic carbon, composite materials, and other suitable natural materials that are biocompatible.

The body material may comprise a material favoring tissue ingrowth, to aid in anchoring the prosthesis to the annulus, and to prevent expulsion of the implanted prosthesis under physiologic loads, once post-surgical healing has occurred. The body material may be flexible in some embodiments, and in some embodiments may have a suitable degree of elasticity. For example, in one embodiment, the body material may be sufficiently pliable to conform to end plate surfaces, and may have sufficient tensile strength to allow for pressurized restoration of disc space height. In one embodiment, the body material may be substantially impermeable. For example, an substantially impermeable body material may prevent leakage of a filler material when an expansible body prosthesis is distended under pressure caused by relative movement of the adjacent vertebrae.

While some embodiments of prostheses according to the present invention may comprise a single expansible body, one embodiment may comprise an expansible body comprising two or more compartments might be employed to allow for more sophisticated applications. For example, a first expansible body may be placed within a second expansible body, wherein the first expansible body may be filled with a hardenable filler material that retains viscoelastic properties and the surrounding second expansible body may be filled with a hardenable material that is less elastic or non-elastomeric. Use of a compartmentalized expansible body prosthesis may allow for close approximation of the functional properties of a healthy natural human intervertebral disc.

In an embodiment wherein the prosthesis comprises an expansible body, the expansible body may be filled with a flowable filler material. A flowable material may comprise, for example, a liquid material, a gaseous material, a slurry, a sludge, a plasma, a paste, a flowable solid (such as powdered, pulverized, granulated, pelletized, or encapsulated material), or any other suitable material that may flow naturally or be made to flow from one place to another. For example, an expansible body may be filled with a saline solution, or a hydrogel and water mixture, so as to produce a prosthesis comprising properties similar to those of a healthy natural intervertebral disc nucleus.

In some embodiments, the flowable filler material may be configured to at least partially solidify after it has flowed into an expansible body prosthesis. For example, polymethyl methacrylate, polycarbonate, plastics, various polymerizing resins or any suitable biocompatible material capable of being injected in a liquid or semi-solid state and then capable of assuming or retaining a functional structural shape or property may be employed as a filler material in one embodiment.

An expansible body prosthesis may comprise an aperture configured to receive a flowable filler material therein. The aperture may comprise a valve, or may be provided as part of a valve structure. For example, an internal valve such as a flap valve may be employed. A different embodiment may comprise an external valve body.

The aperture or the valve may be fashioned from either an imaging transparent material, for example titanium, carbon fiber, or a durable biocompatible plastics material such as polypropylene, or a radiopaque material. In one embodiment, the valve structure may include a one way valve arrangement which may comprise a flap valve partially attached to an inside of the expansible body, such that the valve structure will be in a closed state when an internal pressure of the expansible body exceeds an exterior or injection pressure of a flowable filler material. In another embodiment, a one-way valve may comprise a conical nose with a narrower internal opening. Such a conical configuration may facilitate insertion of various elements, for example, strengthening elements, into the expansible body either prior to or after the expansible body has been at least partially filled with a flowable filler material.

In yet another embodiment, a valve structure may comprise a valve body with a longitudinal bore therein, and an obturating body (such as a ball valve member fabricated from polytetraflouro-ethylene or another suitable material) associated with the bore. Such a valve structure may further comprise a biasing element, such as a spring fabricated from stainless steel or nickel, in communication with the biasing element and the valve body. The valve body may be configured to be secured to the edge portion of the expansible body. For example, an exterior portion of the valve body may be attached to the edge portion of the expansible body such that the longitudinal bore is in communication with an interior of the expansible body. The obturating body may be configured to selectively allow passage of a flowable filler material, such as a saline solution, silicone, or a hydrogel through the longitudinal bore. The valve body may be generally symmetrical and the longitudinal bore may extend axially within the valve body.

An expansible body may comprise one or more strengthened portions. The strengthened portion may comprise a radiopaque marker for indicating the position or orientation of at least a portion of the strengthened portion, and thus of the expansible body. A strengthened portion may be configured to help reduce a risk of further damage to an adjacent annulus or movement by at least a portion of a prosthesis through the annulus. A strengthened portion may or may not have any axis of symmetry with the expansible body prosthesis on which it may be found. As such, in one embodiment, an expansible body prosthesis may comprise an anisotropic configuration.

An expansible body prosthesis according to one embodiment of the present invention may comprise a first substantially impermeable portion having a first structural stiffness. Such an expansible body may further comprise a second substantially impermeable portion contiguous to the first substantially impermeable portion. The second substantially impermeable portion may have a second structural stiffness greater than the first structural stiffness. The second substantially impermeable portion may thus comprise a strengthened portion of the expansible body, and the first substantially impermeable portion may comprise an unstrengthened portion of the expansible body. The second substantially impermeable portion may be configured to be disposed adjacent a tissue in a treatment area when the expansible body is expanded.

When implanted within an annulus of an intervertebral disc, a prosthesis device may be subject to varying forces, pressures, and stresses as a result of a relative movement between the adjacent vertebrae. A strengthened portion may comprise a different structural stiffness than a non-strengthened portion of a prosthesis, and may be configured to restrict expansion of the prosthesis in one or more directions near the strengthened portion, thereby protecting adjacent tissue in a treatment area. For example, the strengthened portion may be configured to provide greater structural stiffness, and thereby permit less displacement under load, near a portion of an annulus that is identified as having been previously weakened by deformity, an injury, or a surgical procedure. As such, a physician may be able to select or modify a prosthesis based on the unique physical anatomy or activities of each individual patient, tailoring the prosthesis to the individual patient.

For example, in one embodiment, a prosthesis comprising a strengthened portion may be implanted as a result of a herniated disc injury. The offending portion of the nucleus may have passed through a portion of the annulus adjacent the spinal cord, causing pressure thereon, and possibly generating pain or numbness in the patient. In such a circumstance, a physician may choose to use an expansible body prosthesis comprising a strengthened portion that is configured to abut the portion of the annulus that the herniated nucleus material passed through. The strengthened portion may be configured to abut a natural tissue that a physician has reason to believe is deficient in its ability to perform its required function. In one such embodiment, the strengthened portion may be configured to be disposed adjacent the identified tissue at least when the expansible body is in an expanded state within the annulus.

Under load, the strengthened portion may comprise a smaller displacement than other non-strengthened or less-strengthened portions of the balloon. By implanting such a prosthesis, the chances of at least a portion of the prosthesis passing through the annulus in the same location may be reduced. Such a strengthened portion may also serve to keep a flowable filler material used to fill the prosthesis contained within the prosthesis.

In one embodiment, an expansible body prosthesis may be provided, wherein the entire expansible body comprises a strengthened portion. However, it may provide greater mobility to a person in whom the prosthesis is implanted if at least a first portion of the expansible body prosthesis is less structurally stiff than a second portion thereof. For example, the natural annulus in many living creatures is not uniformly structurally stiff. Accordingly, to more closely approximate the range of motion provided by a natural annulus, a user may wish to strengthen only selected portions of a prosthesis.

In one embodiment, an expansible body prosthesis may comprise a strengthened portion configured to approximate the properties of a valve or other apparatus located adjacent an aperture used to fill the expansible body prosthesis. In such an embodiment, the strengthened portion may help eliminate asymmetrical resistance to movement that may be caused by such apparatus at or near an aperture. The strengthened portion may accordingly comprise an analog of the aperture when the aperture is sealed.

A strengthened portion may be provided on an expansible body prosthesis using a variety of suitable methods according to the present invention. In some embodiments, a strengthened portion may be provided prior to delivery of the prosthesis device to a user. For example, the strengthened portion may comprise an area of thicker, stiffer, reinforced, or cross-linked material.

In one such embodiment a glass reinforced epoxy coating material may be coupled or otherwise applied to a portion of an expansible body prosthesis' body material, thereby strengthening at least a portion thereof. A coating material may be applied to one or both of the interior and exterior surfaces of a portion of an expansible body prosthesis, thereby strengthening the portion to which it is applied.

In another embodiment, an expansible body prosthesis may be fashioned from a single body material, but may comprise a variety of thicknesses. Relatively thicker portions may be subject to a lesser degree of displacement under load, and as such may comprise strengthened portions of the expansible body. Such an embodiment may be fabricated using one or more of an extrusion process, a hot or cold molding process, or an injection molding process. A biocompatible material may be extruded into the desired configuration, for example, or may comprise additional coatings of the material applied in sequence to select portions of the prosthesis.

In yet another such embodiment, a fibrous material, or a biocompatible cloth may be coupled to a portion of an expansible body prosthesis to strengthen that portion, thereby acting as a brace. A brace may be coupled to one or both of the interior and exterior surfaces of an expansible body prosthesis to provide one or more strengthened portions thereof.

An expansible body prosthesis according to some embodiments of the present invention may comprise a plurality of different body materials. For example, in one such embodiment, a first substantially impermeable portion of an expansible body may comprise a first body material, and a second substantially impermeable portion of the expansible body may comprise a second body material. The second body material can thusly provide the second substantially impermeable portion of the expansible body with a structural stiffness greater than the structural stiffniess of the first substantially impermeable portion.

In other embodiments, a strengthened portion may be provided by a user of the device either prior to, during, or sometime after a surgical procedure used to implant the prosthesis. In one such embodiment, a user may couple a stiffening structure, such as a titanium plate brace, to either the inserted prosthesis, or to a body structure, such as an annulus. In another such embodiment, a physician may apply a stiffening agent coating material, such as a glass reinforced epoxy to the expansible body prosthesis, for example by brushing the glass reinforced epoxy material onto the prosthesis.

In other embodiments, at least a portion of an expansible body prosthesis may comprise a body material comprising a variable structural stiffniess. For example a portion of an expansible body may comprise a body material configured to be crosslinked by exposure to an energy source, such as electron beam radiation or ultraviolet light. Some cross-linkable materials, for example, comprise phenol formaldehyde resins, epoxy resins, amino resins, polyurethanes, and unsaturated polyesters. A prosthesis may be fashioned from a material configured to be crosslinked, while still retaining at least one of its pre-crosslinked properties. In such embodiment, a user may be able to add to or otherwise adjust or vary the structural stiffness of the cross-linkable portion by applying an energy source thereto. The energy source may be applied by a manufacturer of the expansible body, by a user prior to a surgery based on a patient's physiology, during a surgery, or after a surgery while the expansible body prosthesis is implanted in a treatment area. The energy source may be applied either before or after the surgical site has been closed in various embodiments.

The expansible body may be inserted into a cavity provided in the nucleus of an intervertebral disc following removal of at least a portion of the nucleus. The expansible body may be configured such that it is configured to fit the shape of the cavity provided within the annulus. The expansible body may be inserted into such a cavity in a contracted state. For example, in one embodiment, the expansible body may be evacuated and then rolled or folded to facilitate movement along a path established by a cannula prior to use.

An introducing apparatus, such as a tubular elongate member, may facilitate orientation of an expansible body intervertebral disc prosthesis. Such an elongate member may have an internal channel and an aperture at or adjacent its distal end for communicating with the channel. The elongate member may be configured to permit a flowable filler material to flow through the internal channel and into the expansible body.

Introduction of a flowable filler material into the expansible body may cause the expansible body to unfold and expand to fill a nuclear cavity. An expansible body prosthesis may remain in position due to a snug fit within the annulus, and by virtue of the prosthesis being considerably larger after it has been at least partially filled with a filler material than a hole in the annulus through which the prosthesis was inserted. Also, adhesions may develop between the prosthesis and the annulus over time.

Strengthened portions of an expansible body prosthesis may be configured to be adjacent weakened portions of the annulus (comprising areas weakened during implantation of the prosthesis, and areas weakened by aging, degeneration, or traumatic or other injury) when the expansible body has been properly positioned within the annulus. A physician can maneuver an expansible body prosthesis within a treatment area to position the strengthened portion adjacent the identified weakened annulus tissue. These strengthened portions may serve to lessen risks associated with the prosthesis rupturing or herniating the annulus in the weakened areas of the annulus.

When a filler material is injected into the expansible body under pressure, it may pressurize the expansible body against the annulus, thereby helping to prevent the expansible body from escaping through any weaknesses or tears in the disc annulus that may be present.

Any suitable flowable filler material may be used, for example polyvinyl alcohol, a saline solution, or silicone. In another embodiment, the filler material may comprise a hydrogel, for example a polyvinyl alcohol material, such as HYPAN®, developed into a fluid or liquid form which will flow through the valve structure and subsequently harden. In one such embodiment, the swelling pressure of the resultant hydrogel may be similar to that of a healthy lumbar intervertebral disc.

An expansible body may be sized such that an internal surface area and configuration of a nuclear cavity is substantially the same as the expansible body prosthesis. This may enhance load distribution within the implanted prosthesis, providing a load distribution similar to that of a healthy normal intervertebral disc.

In one embodiment, the expansible body may include a deformable porous body shaped to fit within the expansible body. Such a porous body may be formed, for example, from a fine mesh, a loose roll, or an artificial or natural sponge material. In one embodiment comprising a porous body, the porous body may be fashioned from a polypropylene or another suitable material. A porous body may be formed in such a way that it may be inserted into the expansible body through the aperture therein either before the expansible body is inserted into a disc nucleus cavity, or when the expansible body has been positioned by a user in a disc nucleus. The porous body may be attached to the expansible body. A porous body may serve to alter a property of a flowable material inserted into the expansible body such that it will not flow as a liquid, but will deform in a way more similar to the natural disc nucleus.

A porous body may be inserted into the expansible body prior to injection of a flowable filler material. In one embodiment, a fine wire of a radiolucent or radiopaque material may be incorporated within the expansible body prosthesis to demonstrate the position of a prosthesis in vivo.

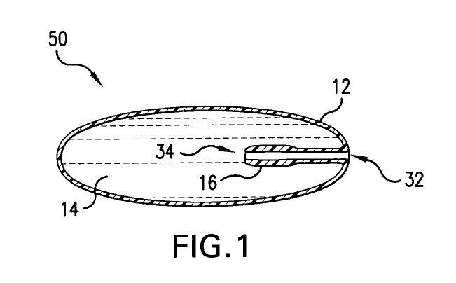

Referring now to the Figures, in which like part numbers depict like elements throughout the Figures, FIG. 1 is cross-sectional elevation view of an expansible body prosthesis 50 according to one embodiment of the present invention, shown in an expanded state and showing an integral, internal one-way fill valve 16. The prosthesis 50 comprises a body material 12 that is configured to approximate the shape of the nucleus pulposus when expanded with a filler material 14. Body material 12 may comprise an inert material such as silicone or a similar elastomer, or a biodegradable and biocompatible material such as poly (DL-lactic-co-glycolic acid; PLGA). The body material 12 may comprise an substantially impermeable material to restrict movement of the filler material 14 therethrough.

The prosthesis 50 may be configured to serve as an artificial inner annulus, and its internal chamber may be configured to contain a pressurized nuclear filler material 14 used for expansion. In one embodiment, the expansible body material 12 may be substantially impermeable while possessing the necessary compliance and strength to approximate the function of a healthy intervertebral disc nucleus. In addition, the body material 12 should be sufficiently flexible so that the implant can easily be passed through a surgical catheter or cannula for insertion.

The prosthesis 50 may be fashioned from a variety of suitable body materials that are biocompatible. In some embodiments, body materials for the prosthesis 50 may be strong and flexible. For example, the prosthesis 50 may comprise a body material comprising one or more plastics, such as flexible PVC (polyvinyl chloride), cross-linked polyethylene, PET, nylon, or Mylar, or may comprise latex or silicone.

In some embodiments, the prosthesis 50 may be coated with a material to aid in its use. For example, the prosthesis 50 may be coated with silicone or Teflon for lubrication, for abrasion resistance, or may be coated with a therapeutic material, such as an anticoagulatory drug. In other embodiments, the prosthesis 50 may comprise a body material that is rupture-resistant or that substantially resists adhesion to a biocompatible filler material.

The prosthesis 50 may be fashioned using a variety of methods known in the art. For example, the prosthesis 50 may be fashioned by dipping a suitably configured mold into a vat of body material in liquid form, and then allowing the body material to change to a solid phase. In another embodiment, the prosthesis 50 may be formed by extruding material into a tube shape, and then forming the tube into a balloon through blow molding. For example, raw materials in granulated form may be heated to melt and liquefy them, and a rotating screw may mix the materials into a homogeneous blend. The liquid material may then be pumped through an extrusion device to extrude the prosthesis 50. The extruded material may then be pulled by a mechanical puller through a cooling bath, freezing it in a solidified form. A mechanical cutter may be used to chop the tubing to its specified length.

The expansible body prosthesis 50 may then be formed from the extruded material through blow molding using a variety of different sizes of glassforms, each configured to provide a prosthesis 50 of a suitable size and shape. One end of the prosthesis 50 may then be welded shut. The finished prosthesis 50 may be inspected for quality control, sterilized, and collapsed using a vacuum pump. The peripheral surface of the prosthesis 50 may be coated with one or more bioactive substances that may promote healing of the inner annulus and integration of the prosthesis 50 with the surrounding annular tissue. Also, the top and bottom surfaces of the implant may be coated with one or more bioactive substances that may promote healing of the cartilaginous endplates of the adjacent vertebrae and integration of the prosthesis 50 with the endplates.

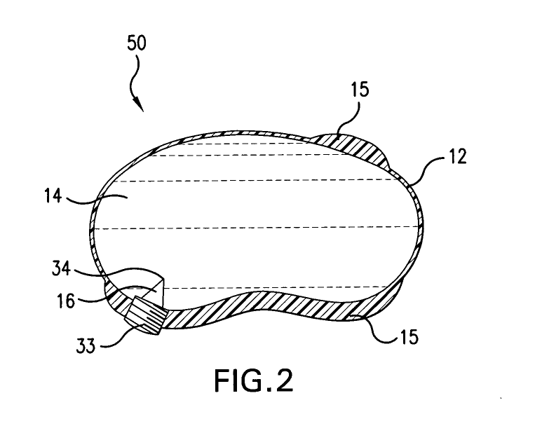

To limit the amount of lateral bulging when the prosthesis 50 is axially compressed, the prosthesis 50 may comprise one or more strengthened portions (see FIG. 2) as described above. For example, a peripheral surface of the prosthesis 50 can be reinforced with a fiber matrix brace to provide a strengthened portion if desired. Prosthesis 50 comprises an integral, internal, self-sealing, one-way valve 16 that may allow the prosthesis 50 to be inserted in a contracted state and then be expanded in situ with a lesser risk of contraction. Valve 16 functions as a flapper valve to prevent leakage and maintain pressurization of the prosthesis 50 when pressurized with the nuclear filler material 14. Compression of expansible body prosthesis 50 may place internal pressure on valve 16 to keep it in a closed position. Due to the self-sealing nature of valve 16, the same pressure that might be sufficient to allow the nuclear filler material 14 to escape may cause valve 16 to remain closed so as to create a barrier to extrusion.

To inflate the implant, a needle-like fill stem may be inserted through entrance port 32 so as to puncture the distal end 34 of valve 16 and extend into the interior chamber of the implant. The implant may then be filled with a flowable filler material 14, such as a high molecular weight fluid, a saline solution, silicone, gel or combination of fluid and elastomer which has a viscosity that may permit its introduction into the prosthesis 50 through, for example, an 18-gauge needle. The specific properties of filler material 14 may allow the material to achieve and maintain the desired osmotic pressure. The filling may take place after the prosthesis 50 is placed within an intervertebral disc. The filler material 14 may comprise a cross-linkable polyethylene glycol (PEG) hydrogel with chondroitin sulfate (CS) and hyaluronic acid (HA) with or without host cells.

Referring now to FIG. 2, a cross-sectional plan view of a prosthesis 50 according to one embodiment of the present invention is shown in an expanded state. The prosthesis 50 is coupled to a threaded fitting 33 and a conical nose one-way valve 16 coupled to an aperture through the body material 12 of the prosthesis 50. The body material 12 of the prosthesis 50 comprises a plurality of strengthened portions 15. The strengthened portions comprise areas of thicker body material 12. The strengthened portions 15 are configured to abut portions of an annulus that may be prone to retropulsion or anteropulsion of the prosthesis 50. Such areas may be the result of deformity, aging, injury, or surgical procedure, for example. The larger of the two strengthened portions 15 shown in FIG. 2 is configured to abut the posterior side of the annulus, thereby helping to prevent retropulsion of the prosthesis 50 through the annulus.

The threaded fitting 33 may be configured to receive a distal end of an elongate member (not shown) that may be used to position the prosthesis 50 within a treatment area. The filler material 14 may flow through a channel in such an elongate member, into the entrance port 32 through the one-way conical nose valve 16, exiting through the distal end 34 of the valve 16 and into the interior of the expansible body prosthesis 50. For example, in one embodiment, an elongate member may be coupled with the prosthesis 50 by screwing a threaded distal end of the elongate member into the threaded fitting 33 prior to inserting the prosthesis 50 into a treatment area. Upon satisfaction that the expansible body prosthesis 50 has been properly oriented and expanded in the treatment area, a surgeon may uncouple and remove the elongate member from the surgical site.

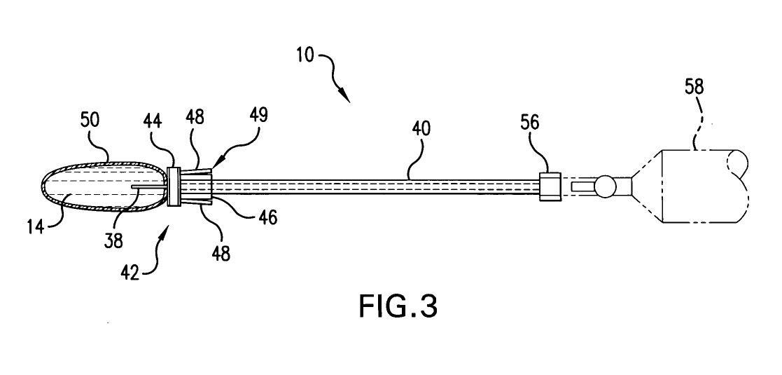

Referring now to FIG. 3, an assembled elevation view of a delivery system 10 for placement of a prosthesis 50 according to one embodiment of the invention is shown. The delivery system 10 comprises a hollow implant fill stem 38, a hollow elongate member 40, and an inner annular buttress 42. The implant fill stem 38 is configured to conduct a flowable material 14 into the prosthesis 50 after it has been inserted into a cavity formed by removal of nuclear material from within an annulus.

The system 10 shown in FIG. 3 further comprises the elongate member 40 configured to be maneuverable along a path established by a cannula. The elongate member 40 may be made from a resilient inert material providing torsion transmission capabilities (e.g., stainless steel, a nickel-titanium alloy such as Nitinol, and other suitable metal alloys). In other embodiments, the elongate member 40 may be fashioned from a variety of suitable materials, comprising a carbon fiber, a glass, or a flexible material, such as a plastic or rubber. In one embodiment comprising a flexible elongate member 40, the elongate member 40 may be, for example, fashioned from twisted wire filaments, such stainless steel, nickel-titanium alloys (such as Nitinol), and suitable other metal alloys. The elongate member 40 shown is hollow, allowing movement of a flowable filler material 14 within a bore therethrough along its axis.

The prosthesis 50 can, in cross section, be polygonal, rectilinear, ovoid, asymmetrical, d-shaped, kidney bean shaped, or any other suitable configuration. While in a collapsed state, the prosthesis 50 comprises a collapsed dimension, thereby fitting within the inside bore dimension of a cannula, allowing the prosthesis 50 to be maneuvered along a path established by the cannula. Such a clearance may allow a user of the system 10 to maneuver the elongate member 40, and thereby the prosthesis 50, within and along the axis of the cannula. The elongate member 40 and the prosthesis 50 are configured to be rotated if the user so desires.

The prosthesis 50 may comprise a predetermined expanded dimension and shape. In one embodiment, a user of the system 10 may select the prosthesis 50 from a plurality of selectable prostheses 50, each comprising a unique predetermined expanded dimension or shape. In a different embodiment, the prosthesis 50 may not comprise a predetermined expanded dimension or shape, and may be configured to expand to fill a cavity provided therefore. A user in such an embodiment may select the prosthesis 50 based, at least in part, on a configuration of tissues in the treatment area.

In another embodiment of the present invention, at least a portion of the prosthesis 50 or the elongate member 40 may comprise one or more radiological markers. A radiological marker may permit radiologic visualization of at least one of the elongate member 40 and the prosthesis 50 within a treatment area. The markers may be fashioned from a radiopaque material, such as platinum, gold, calcium, tantalum, and other heavy metals. In other embodiments, other forms of markers can be used to allow a user to visualize the location, size, and shape of at least the prosthesis 50 within the treatment area. For example, the prosthesis 50 may be expanded with a radiopaque gas or liquid.

A system according to one embodiment of the present invention, such as the system 10 described with respect to FIG. 3, can comprise an interior lumen. The lumen may be coupled, for example, to an external source of a flowable material and an external vacuum source. In one such embodiment, a rinsing liquid, e.g., sterile saline, can be introduced from the source through the lumen into the treatment area before, during or after insertion of the prosthesis 50 into a cavity in the intervertebral disc. A rinsing liquid may reduce friction and conduct heat away from the tissue. The rinsing liquid can be introduced continuously or intermittently while the tissue mass is being compacted, removed, or cut. The rinsing liquid can also carry an anticoagulant or other anti-clotting agent. In one such embodiment, the lumen may be coupled to the vacuum source, and liquids and debris can be aspirated from the tissue region through the lumen.

The inner annular buttress 42 shown in FIG. 3 is configured to extend into and block a hole provided in the annulus for insertion of prosthesis 50. The inner annular buttress 42 may prevent extrusion of the prosthesis 50 or the filler material 14 therein during spinal loading. The inner annular buttress 42 may comprise a polymer head portion 44 of suitable diameter for plugging a hole provided in the annulus. The inner annular buttress 42 further comprises a smaller diameter polymer body portion 46 extending from head portion 44.

The inner annular buttress 42 further comprises barbs 48 having ends 49 that are configured to extend outward in relation to the body portion 46. The ends 49 of the barbs 48 are configured to engage the annulus tissue to prevent expulsion of inner annular buttress 42, and thereby prosthesis 50 during spinal loading. The barbs 48 can, for example, be formed of stainless steel, Nitinol®, a polymer, or another suitable biocompatible material, and can be molded into or otherwise coupled with the head portion 44 for retention therein.

An inner passage extends through the inner annular buttress 42 for attachment to the elongate member 40 and insertion of the fill stem 38. The fill stem 38 may be inserted through the inner annular buttress 42 into the prosthesis 50. The inner passage, the head portion 44, and the body portion 46 may be coaxial in configuration. The elongate member 40 and the inner annular buttress 42 are coupled together using mating threads 54a, 54b or another suitable form of detachable coupling that may allow the elongate member 40 to be easily removed from inner annular buttress 42 after placement in a treatment area. For example, in one embodiment, the elongate member 40 may be fashioned from a continuous piece of material with the buttress 42, and may be scored in an area near the buttress 42. A user of such a device may break the two elements apart by pulling or twisting the elongate member 40. The inner annular buttress 42 can be coupled to the prosthesis 50 using adhesives, ultrasonic welding, or the like, or can be separate and unattached from the prosthesis 50.

Fill stem 38 comprises a collar 56 for attachment to a syringe 58 or other device to be used for expanding the prosthesis 50 with the filler material 14. The fill stem 38 and the syringe 58 may be coupled together using threads (not shown) or another form of detachable coupling. The syringe 58 may comprise a pressure gauge (not shown) for determining the proper interior pressure of the prosthesis 50. The prosthesis 50 and the delivery system 10 may be deployed into a cavity formerly occupied by nucleus pulposus material. Access to such a cavity may be provided by a conventional catheter, cannula or the like (not shown). In one embodiment, a cannula may comprise a retractable cover (not shown) configured to protect the prosthesis 50 during insertion into the treatment area.

The system 10 shown in FIG. 3 may comprise a controller (not shown). At least some portion of the expansible body prosthesis 50 or the elongate member 40 may be in communication with one or more suitable types of controller, such as a slide controller, a pistol grip controller, a ratcheting controller, a threaded controller, or any other suitable type of controller that can be configured to permit an operator of the system 10 to control at least one of the extent to which the prosthesis 50 extends beyond a distal end of a cannula, or the extent to which it is expanded or contracted.

For example, a controller may be configured to allow an operator of the system 10 to adjust the volume of filler material 14 in the prosthesis 50. By adjusting the volume of filler material in the prosthesis 50, an operator of the system 10 may expand or contract the prosthesis 50. In one embodiment of the present invention, a controller can also comprise indicia by which the physician can visually estimate the extent to which a controlled element has been adjusted.

In one embodiment, a suction tube may also be deployed along a path established by a cannula to remove material from the interior of the intervertebral disc when a cavity in the nucleus therein is provided. In yet another embodiment, the system 10 may comprise an interior lumen to serve as a suction tube as well as to convey a rinsing liquid into the treatment area as cavities are being formed or filled therein. The suction tube (or a lumen) may introduce a rinsing fluid (with an anticoagulant, if desired) and may remove loosened tissue. Alternatively, a cannula may comprise a first interior lumen that serves as a suction tube, and a second interior lumen that serves to flush the treatment area.

Another suitable tool can be deployed through a cannula into the treatment area. For example, in one embodiment, another tool may, for example, perform a diagnostic or therapeutic procedure (such as providing a therapeutic material to the tissues in the treatment area). In one embodiment, at least a portion of the prosthesis 50 may be coated with a therapeutic material. When inserted into the treatment area, the expansible body may thus provide a therapeutic effect to a tissue therein by contacting the tissue and depositing some of the therapeutic material thereon. For example, an allograft material, a synthetic bone substitute, a medication, or a biocompatible flowable material that may set to a hardened condition may be provided to some portion of a treatment area by an expansible body according to one embodiment of the present invention.

In one embodiment, the system 10 may also be used to apply radiation therapy or chemotherapy. Further details of the injection of such materials into a treatment area for therapeutic purposes may be found in U.S. Pat. Nos. 4,3969,888 and 5,108,404, and in co-pending U.S. patent application Publication No. 2003/0229372, which are incorporated herein by reference.

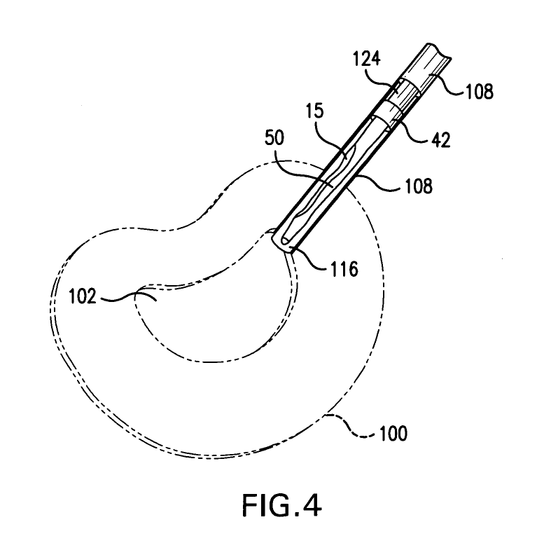

FIGS. 4-7 are perspective views of a human intervertebral disc 100, wherein a cavity 102 formerly occupied by the nucleus thereof is being accessed by a system configured to insert a prosthesis 50 comprising a strengthened portion 15 according to one embodiment of the present invention. As shown in FIG. 4, prior to deployment of the prosthesis 50 in the nuclear cavity 102, the prosthesis 50 is contracted and housed within an introducer sheath 108.

The introducer sheath 108 may be used as a port or cannula into the nuclear space 102. As shown in FIG. 4, the distal end portion of the introducer sheath 108 has been cut away for clarity. A plastic launcher sheath 116 is shown within the introducer sheath 108. The launcher sheath 116 is configured to be slidably insertable into the introducer sheath 108. The distal end portion of the launcher sheath 116 has also been cutaway for clarity. The launcher sheath 116 may comprise a handle to aid an operator thereof, and all or a portion of the launcher sheath may be flexible to assist with deployment of the prosthesis 50.

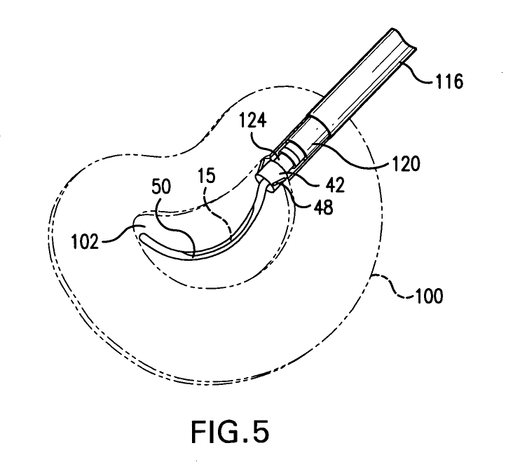

A fill tube 120 (shown in FIGS. 5-7) may be used to provide a filler material into the interior of the prosthesis 50. The fill tube 120 is configured to be slidably insertable into the launcher sheath 116. The fill tube 120 also may comprise a handle configured to allow an operator to manipulate the fill tube 120. The fill tube 120 may terminate at its distal end with a female leur lock 124 to which the assembly of a buttress 42 may be threadably coupled. The buttress 42 may be permanently or temporarily coupled to the prosthesis 50.

The buttress 42 can be attached to the leur lock 124 after the fill tube 120 has been inserted into the launcher sheath 116 and extended therethrough such that the leur lock 124 extends through the end of the launcher sheath 116. The buttress 42 comprises a plurality of barbs 48 (as shown in FIG. 5-7). The barbs 48 can be manually depressed and the undeployed implant/buttress assembly can be pulled into the launcher sheath 116. Alternatively, the buttress 42 can be attached to the leur lock 124 and the fill tube 120, prior to insertion into the launcher sheath 116. The assembly of the prosthesis 50, the buttress 42, the launcher sheath 116 and the fill tube 120 can then be inserted into the introducer sheath 108 and pushed into the nuclear space 102.

Referring now to FIG. 5, a perspective view of the assembly of FIG. 4 is shown after deployment of the prosthesis 50 in the nuclear cavity 102, and prior to retraction of the leur locked and barbed buttress 42 thereof into the annulus tissue of the intervertebral disc 100.

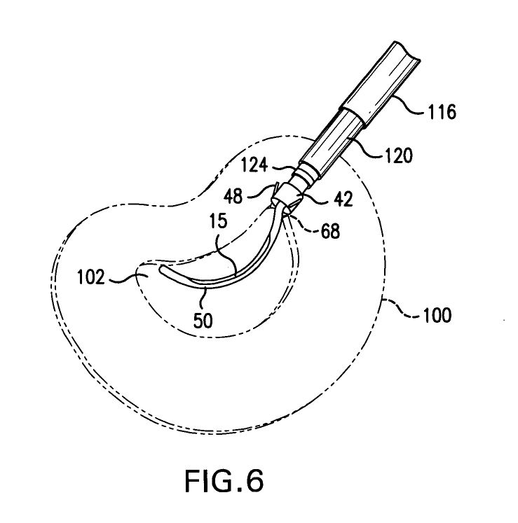

FIG. 6 is a perspective view of the assembly of FIG. 4 after retraction of the threaded and barbed buttress 42 into the annulus, and plugging the annular opening in the intervertebral disc 100.

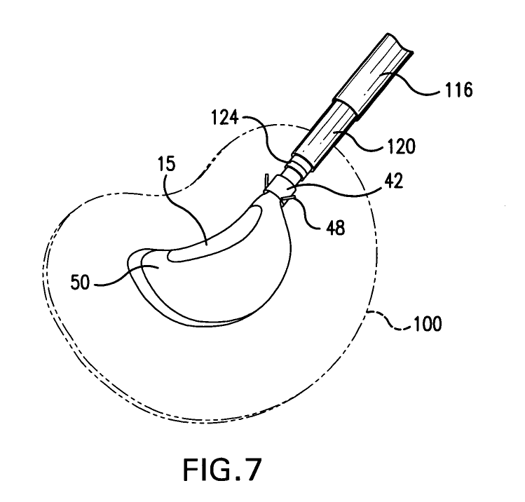

FIG. 7 is a perspective view of the assembly of FIG. 4 after the prosthesis 50 has been expanded in the nuclear cavity 102 by provision of a biocompatible filler material therein. As shown in FIG. 7, the strengthened portion 15 abuts the posterior wall of the annulus, providing structural support thereto, and helping to resist against retropulsion of the prosthesis 50.

It should be appreciated, that systems and methods according to the present invention are not limited in application to human intervertebral discs, and may be used to replace or augment tissue in other parts of a living or non-living organism. For example, the system 10 shown in FIG. 3, or the prosthesis 50 shown in FIG. 4-7 can be deployed in other embodiments in other joints in other tissue types of the human body, such as in an elbow or knee joint, etc.

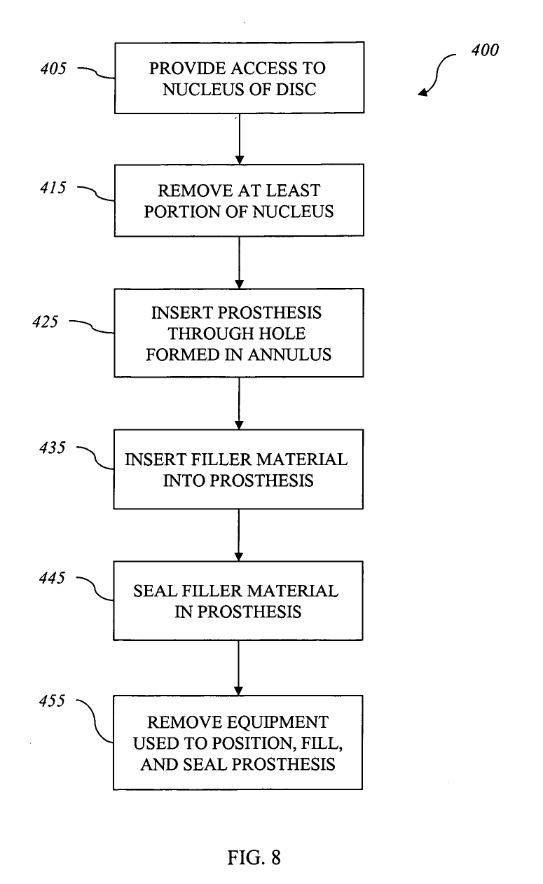

Referring now to FIG. 8, a flow chart of a method 400 according to one embodiment of the present invention is shown. The method 400 comprises a method of insertion of a prosthesis. The method 400 comprises providing access to a nucleus of a damaged intervertebral disc, as shown in box 405. For example, a surgeon may make a small skin incision with the point of a knife adjacent a damaged intervertebral disc, including an annulus and a nucleus. The disc may have previously been extensively imaged by a Computer Tomography (CT), using Magnetic Resonance Imaging (MRI), X-ray photography, or other suitable device. If necessary, confirmation that the subject disc is the disc that is painful may be reached by effecting provocative stress discography.

The incision may allow percutaneous disc surgery to be carried out by a lateral approach whereby an operating port and trochar may be used to insert instruments laterally between adjacent vertebrae in the spine through the paraspinal musculature so entering the disc at the postlateral corner in the safe triangle, inferior to the exiting nerve root. The trochacr may create a hole in the annulus by spreading the fibers of the annulus and provides for access to the nucleus portion of the intervertebral disc.

In one embodiment, a lateral percutaneous approach to the injured disc is used. After an incision is made in the skin at the appropriate point, and under imaging control, a needle may be passed to the posterolateral point of the disc's annulus. Tissues may then be dilated by a series of dilating tubes, and finally a cannula can be passed down to the level of the annulus. The dilators may then be removed, leaving an operating channel. A hole may then be imparted through the annulus to access the nucleus, and a needle of appropriate bore may be used to enter the nucleus via the hole provided in the annulus.

The method 400 further comprises removing at least a portion of the nucleus of the accessed intervertebral disc, as shown in box 415. A physician may determine what amount, if any, of a herniated or ruptured disc's nucleus material should be removed, either prior to, or during a surgical procedure. In one embodiment, substantially all of the nucleus may be removed. In a different embodiment, only a herniated portion thereof may be removed.

In one embodiment, a drug, such as chymopapain, may be injected into the nucleus to digest the proteoglycan structure thereof. This may be done either prior to the creation of the hole in the annulus or through such a hole after it has been provided. Mechanical action as by a brush with polypropylene bristles may be used to aid the breakdown of any remaining collagen structure to enhance the effect of chymopapain that may then be removed by suction. In a different embodiment, a curette may be used to scrape out the desired portion of the nucleus. Such scrapings may then be removed by suction. In yet another embodiment, at least a portion of the nucleus may be removed piecemeal by rongeurs.

In another embodiment, the annulus may be laser annealed after removal of at least a portion of the nucleus. Laser annealing may cause shrinking and tightening of the tissues thereof to reduce the size of any lateral or posterior tears in the annulus from which some of the nucleus pulposus may have expressed out from the intervertebral disc space.

The method 400 further comprises inserting an intervertebral disc nucleus prosthesis through the hole formed in the annulus, as shown in box 425. The disc's annulus comprises an outer ring of strong collagenous fibrous tissue. The prosthesis may comprise, for example, an expansible body having a configuration shaped to fit within the annulus in a cavity formed by removal of at least a portion of the nucleus.

In one embodiment, substantially all of the nucleus may have been removed from the annulus. In such an embodiment, an intervertebral disc prosthesis may comprise an expansible body manufactured to closely approximate the shape and dimension of a healthy natural nucleus. In an embodiment where only a portion of the nucleus material has been removed, a prosthesis may be configured to abut the remaining nucleus material and the adjacent annulus.

An expansible body prosthesis may be evacuated and folded or rolled prior to insertion such that is maneuverable along a path established by a cannula and through a hole provided in the annulus into the space created by the removed nucleus or portion thereof. An expansible body prosthesis according to the present invention may comprise a strengthened portion or a portion configured to be strengthened, such as a portion of body material comprising a variable structural stiffness, upon a determination by a user that a patient's anatomy requires a strengthened portion as described above.

For example, in one embodiment, at least a portion of an expansible body prosthesis may comprise a body material configured to be crosslinked when exposed to an energy source, such as ultraviolet light. In such an embodiment, a surgeon may determine that the patient's anatomy requires that a strengthened area should be provided on a side of the expansible body facing the portion of the annulus adjacent the neural arch, to protect against retropulsion of the prosthesis. In such a situation, a surgeon may use ultraviolet light to crosslink at least a portion of the body material, thereby providing a strengthened portion.

In some embodiments, an expansible body prosthesis may comprise an external valve structure. In one such embodiment, prior to insertion of the expansible body into a treatment area, the external valve structure of the prosthesis may be permanently or temporarily coupled to an elongate member. The elongate member may comprise a bore axially therethrough, wherein the bore is configured to permit a flowable material to travel therethrough into or out of the expansible body. A user may apply a force to the elongate member, thereby pushing, rotating, or otherwise maneuvering the expansible body prosthesis in the treatment area until the user has determined that it comprises the appropriate location and orientation. For example, a surgeon may maneuver an expansible body prosthesis comprising a strengthened portion to align the strengthened portion thereof with an annulus tissue he or she has identified as being subject to rupture or other new or repetitive injury.

The elongate member may be maneuverable along a path established by a cannula, and may be releasably coupled to the expansible body prosthesis. For example, an expansible body prosthesis comprising a valve structure including a threaded portion may be temporarily coupled to a hollow elongate member using a screw thread coupling. In one embodiment, the elongate member may be attached to a prosthesis prior to insertion of the prosthesis, and the prosthesis may be folded in such a way as to ensure its ease of passage through the annulus into the nucleus. Such an arrangement may be formed and inserted inside a cannula, which would in turn be passed through the operating port to engage the hole in the annulus, and ease passage of the expansible body into the nuclear cavity.

In a different embodiment, the expansible body prosthesis may contain a collapsible sponge, a mesh, or a fine coil which fills at least a portion of the expansible body, and has a memory such that after insertion through the annulus into the disc space, the sponge, mesh or coil will return to its previous shape, i.e. the shape of the nucleus or a cavity therein, and will expand within the expansible body to fill the disc space.

The method 400 further comprises inserting a flowable filler material into the prosthesis, as shown in box 435. A user may insert enough of the filler material into the expansible body prosthesis may expand the expansible body either fully or partially, depending on the situation and conditions. The filler material may comprise a variety of suitable materials, or combination thereof. For example, in various embodiments, the filler material may comprise a composite ceramic paste, a hydrogel material, a saline solution, silicone, water, etc. As described above, a sponge, mesh or fine wire may fill at least a portion of the expansible body, and may already be present within the expansible body, or may be inserted through an elongate member into the expansible body.

In one embodiment, a measured amount of the filler material may be introduced into a syringe. Such a syringe may be long and slender, having a seal at the distal end thereof, and may be introduced through a hollow elongate member coupled to the expansible body. In one such embodiment, a distal end of a syringe may be coupled with a valve structure of an expansible body prosthesis, forming a seal. This may be achieved by a locking mechanism (such as a bayonet fitting) or a threaded connection, for example. A seal between the syringe and the expansible body may help ensure that the filler material passes through the valve structure into the expansible body.

In another embodiment, a filler material may be introduced into the expansible body prosthesis via the a hollow elongate member coupled to the expansible body, for example via a one-way valve. In such an embodiment, the filler material may flow down the internal passage of the elongate member and through a bore in a valve structure and into the unexpanded expansible body prosthesis so as to expand the same. The introduction of the filler material may be continued until the expansible body is adequately filled with the filler material.

In one embodiment, an expansible body prosthesis may comprise a predetermined expanded dimension or volume. In such an embodiment, inserting a filler material into the expansible body prosthesis within a treatment area may comprise filling the expansible body with a predetermined volume of the filler material. For example, a user of the method 400 may select an expansible body prosthesis from a plurality of expansible body prostheses, each comprising a unique expanded dimension or volume. Selection of one of the plurality of expansible bodies may be based, at least in part, on a size or shape of another tool previously inserted into the treatment area, a mass, volume, or other measure of nucleus material removed from the treatment area, results of a magnetic, X-ray, or electronic scan of the treatment area, or a size or shape of a cavity previously provided within the treatment area by another tool.

The method 400 further comprises sealing the filler material in the prosthesis, as shown in box 445. The prosthesis may comprise a valve structure. The valve structure, in one embodiment, may comprise a one-way valve arrangement (or conical nose) and a body that may be knurled or fluted as appropriate. Such a one-way valve may be configured to operate either automatically or manually to prohibit a volume of filler material within an expansible body prosthesis from escaping therefrom.

In a different embodiment, an expansible body prosthesis may, in addition to or in place of a one-way valve, comprise an aperture for insertion of a filler material that is configured to prevent a filler material in the expansible body from escaping therefrom, or to otherwise be sealed, permanently or temporarily, using a different apparatus or method. In some embodiments, the aperture may be configured to passively prevent a filler material from escaping therefrom. For example, at least a portion of an expansible body prosthesis may comprise a silicone self-sealing membrane or a one-way valve.

In other embodiments, the aperture may be configured to be actively sealed. For example, an expansible body prosthesis' aperture may be configured to be sealed, either permanently or temporarily, by crimping, swaging, welding, plugging, or using a biocompatible adhesive or an energy source (such as heat, ultraviolet light or other radiation) to prevent a filler material that has been inserted into the prosthesis from escaping through the aperture. In one such embodiment, a plug may be fashioned from a biocompatible material, such as latex, and may be coupled to the aperture, thereby sealing the filler material within the expansible body, using a biocompatible adhesive. In another embodiment, a heat source may be used to melt a portion of the expansible body surrounding the aperture, thereby preventing a filler material therewithin from escaping through the aperture.

For example, in one embodiment, a syringe used to deliver a filler material into an expansible body prosthesis may comprise a tubular piston rod and a piston that may be selectively secured to a screw configured to immediately close or otherwise plug a valve structure after injection of the filler material. In one such embodiment, once a desired volume of filler material has been inserted into the expansible body prosthesis, and the piston is at the bottom of the syringe, a screwdriver may be passed down a center of the piston to insert and tighten the screw to the valve structure of the prosthesis. In one such embodiment, in the event that the volume of filler material needs to be subsequently altered, this can be performed in a substantially non-traumatic way by merely removing the screw and replacing or augmenting the contents of the expansible body prosthetic as necessary.

In an alternative embodiment, where an external valve structure is not used, a hollow elongate member may pass through a conical valve or a self-sealing membrane of the expansible body prosthesis to at least partially fill it with a filler material. Upon removal from the expansible body, the self-sealing membrane or conical valve may automatically seal due to a higher internal pressure within the expansible body prosthesis than the surrounding body tissue. In another alternative embodiment, a simple flap may be provided over the aperture to act as a one-way valve.

The method 400 finally comprises removing equipment used to position, fill, and seal the prosthesis, as shown in box 455. Such equipment may be removed along a path established by a cannula. In one embodiment, this step may comprise detaching or decoupling a hollow elongate member used to provide the filler material to the interior of an expansible body prosthesis from the expansible body. There are a variety of suitable apparatuses and methods according to the present invention to accomplish this. For example, a portion of the elongate member or the expansible body may be pre-scored to facilitate a material failure or other controlled or uncontrolled breakage in the scored area, thereby separating the expansible body from the elongate member.

In another embodiment, a cutting device may be used, either external or internal to the elongate member, to cut the expansible body from the elongate member. Such a cutting device may be passed, for example, through an interior bore in a hollow elongate member after the expansible body has been sealed, and a user may turn the blade thereof in a complete circle to cut the elongate member from the expansible body from the inside of the elongate member to the outside thereof.

In yet another embodiment, the elongate member may be coupled to the expansible body via a threaded or a bayonet connection, and the elongate member may be rotated by a user to unscrew or unhook the elongate member from the expansible body. In some embodiments, the steps of sealing an expansible body prosthesis and detaching or decoupling it from the elongate member in communication therewith may be simultaneous. For example, in one such embodiment, a frangible coupling may be provided between the expansible body prosthesis and the elongate member. Such a frangible coupling may be configured to rupture upon a predetermined condition. For example, the frangible coupling may rupture when the interior pressure, temperature, or volume of the expansible body reaches a predetermined threshold level. Upon separation from the expansible body prosthesis, the hollow elongate member may be removed from the treatment area along a path established by the cannula.

The hole (or operating port) provided in the annulus may tend to close as the annulus' fibers are stretched. After being filled with the filler material, the prosthesis may be larger in size than the hole provided in the annulus, and may therefore be retained in the disc by the annulus after removal of any equipment used to position, fill, and seal the prosthesis. A strengthened portion of an expansible body prosthesis may be oriented toward the area where the hole in the annulus was provided, providing extra structural strength, thereby helping to protect against movement by either the prosthesis or any remaining nuclear material through the portion of the annulus weakened by providing the hole therein.

In one embodiment, after endoscopic instruments used to perform a procedure have been removed, the skin incision may be closed by suture, staple, bonding agent, adhesive bandage, or like procedure. Some prostheses embodiments according to the present invention may be configured to be removed at a later date. In one such embodiment, after about 1 month to about 3 years, fibrocollagenous tissue may have grown into the intervertebral disc space, a second endoscopic procedure may be performed to remove the expansible body prosthesis.

In such a removal procedure, the filler material may be removed from the expansible body prosthesis, i.e., by opening a ball valve by pushing the valve from the valve seat with an instrument, and the contracted expansible body may then be removed via a cannula. In one such embodiment, the hole provided in the annulus during the procedure used to implant the prosthesis may be maintained by a nubbin (an uninflated portion of expansible body prosthesis device) or a valve body. For example, an imaging technique may be used to facilitate guiding the distal end of the cannula to a radiolucent nubbin.

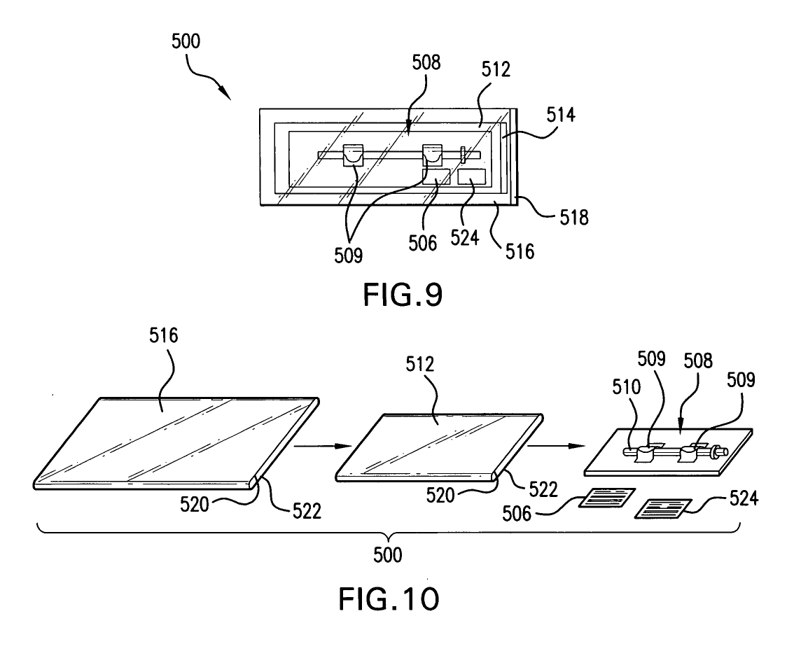

Referring now to FIGS. 9 and 10, a plan view and an exploded perspective view, respectively, of a sterile kit 500 to store a prosthesis and associated surgical tools according to one embodiment of the present invention is shown. At least some parts of a system according to one embodiment of the present invention may be packaged in a sterile kit 500 as shown in FIGS. 9 and 10 prior to deployment in an intervertebral disc or other tissue. In one such embodiment, the tool may comprise a single use tool.

For example, one sterile kit 500 may comprise a selection of access tools (such as needles and various sizes of cannulas), cutting tools (such as curettes and scalpels), an elongate member to introduce an expansible body prosthesis, the prosthesis itself, evacuated and rolled in preparation for insertion into a treatment area through a cannula, a container of a flowable filler material, detaching tools (such as an elongate rod comprising a blade perpendicular to the axis thereof and configured to cut or score the inside of a coupling between an elongate member and the prosthesis or a valve thereof. The sterile kit 500 may further comprise tools that may be required to close up a wound created by a procedure used to implant the prosthesis, such as surgical tape, a sewing kit, etc.

The sterile kit 500 may further comprise one or more tools or substances designed to allow a user to provide a strengthened area to a prosthesis either prior to, during, or after a surgical procedure used to implant he prosthesis. For example, the sterile kit 500 may comprise a tool configured to provide an energy source (such as ultraviolet light) through a cannula to a prosthesis within a treatment area. In one such embodiment, a fiber optic filament may be used to transmit energy along an elongate member to a prosthesis comprising a crosslinkable body material. In another embodiment, the sterile kit 500 may comprise a coating material, or a biocompatible adhesive and one or more structural elements configured to be coupled to the prosthesis in situ or pre-implantation, thereby acting as a brace.

The sterile kit 500 may comprise a plurality of expansible body prostheses, each comprising a unique arrangement of one or more strengthened portions or other unique configurations. In another embodiment, a surgeon may have access to a plurality of different kits, each containing one unique expansible body prosthesis.

As shown in FIGS. 9 and 10, the kit 500 comprises an interior tray 508. The tray 508 holds the system (generically designated 510) in a lay-flat, straightened condition during sterilization and storage prior to its first use. The tray 508 can be formed, for example, from die cut cardboard or thermoformed plastic material. The tray 508 comprises one or more spaced apart tabs 509, which hold the system 510 in the desired lay-flat, straightened condition.

The kit 500 comprises an inner wrap 512 that, in the embodiment shown, is peripherally sealed by heat or the like, to enclose the tray 508 from contact with the outside environment. One end of the inner wrap 512 comprises a conventional peal-away seal 514 (see FIG. 10), to provide quick access to the tray 508 upon use, which may occur in a sterile environment, such as within an operating room.

The kit 500 shown also comprises an outer wrap 516, which is also peripherally sealed by heat or the like, to enclose the inner wrap 512. One end of the outer wrap 516 comprises a conventional peal-away seal 518 (see FIG. 10), to provide access to the inner wrap 512, which can be removed from the outer wrap 516 in anticipation of imminent use of the system 510, without compromising sterility of the system 510 itself.

Both inner and outer wraps 512 and 516 (see FIG. 10) comprise a peripherally sealed top sheet 520 and bottom sheet 522. In the illustrated embodiment, the top sheet 520 is made of transparent plastic film, like polyethylene or MYLAR material, to allow visual identification of the contents of the kit 500. The bottom sheet 522 may be made from a material permeable to ethylene oxide sterilization gas, e.g., TYVEC™ plastic material (available from DuPont®).

In the embodiment shown in FIGS. 9 and 10, the sterile kit 500 also carries a label or insert 506, which comprises the statement “For Single Patient Use Only” (or comparable language) to affirmatively caution against reuse of the contents of the kit 500. The label 506 also may affirmatively instruct against resterilization of the system 510. The label 506 also may instruct the physician or user to dispose of the system 510 and the entire contents of the kit 500 upon use in accordance with applicable biological waste procedures. The presence of the system 510 packaged in the kit 500 verifies to the physician or user that the system 510 is sterile and has not been subjected to prior use. The physician or user is thereby assured that the system 510 meets established performance and sterility specifications, and will comprise the expected configuration when used.

The kit 500 also may comprise directions for use 524, which may instruct the physician regarding the use of the system 510. For example, the directions 524 may instruct the physician to deploy, manipulate, adjust, or otherwise maneuver, the system 510 inside an intervertebral disc to implant a prosthetic device therein.

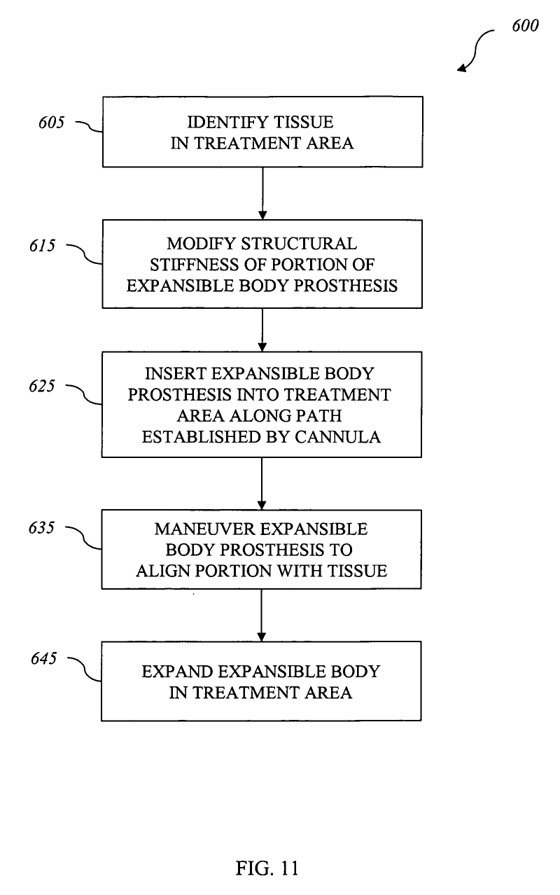

Referring now to FIG. 11, a method 600 according to one embodiment of the present invention is shown. The method 600 comprises identifying a tissue in a treatment area as shown in box 605. For example, a physician may use imaging equipment to identify a weakened, damaged, or defective tissue in an annulus of one or more of a patient's intervertebral discs.

The method 600 further comprises modifying a structural stiffness of a portion of an expansible body. For example, in one embodiment, the expansible body may comprise a prosthesis configured to replace at least a portion of a nucleus in a human intervertebral disc. The expansible body may comprise at least a first portion and a second portion. The second portion of such an expansible body may be strengthened using a variety of suitable methods. For example, in one embodiment, the structural stiffness of the second portion may be modified by increasing the structural stiffness. For example, the structural stiffness of the second portion may be increased in one such embodiment by fabricating the second portion from a different thickness or body material than the first portion of the expansible body.

In another embodiment, coupling a brace to one or both of the interior and exterior surfaces of the second portion may modify the structural stiffness of the second portion. Likewise, in another embodiment, one or both of the interior and exterior surfaces of the second portion may be coated with a material configured to modify the structural stiffness of the second portion. In yet another embodiment, at least the second portion of the expansible body in which the structural stiffness is modified comprises a cross-linkable material. In such an embodiment, cross-linking the second portion may increase the structural stiffness thereof.

Referring still to FIG. 11, the method 600 further comprises inserting the expansible body into the treatment area along a path established by a cannula as shown in box 625. The structural stiffness of a portion of the expansible body may be modified prior to, subsequent to, or while inserting the expansible body into the treatment area. In one embodiment, the expansible body may be inserted into a treatment area in a cavity provided within the nuclear space of a human intervertebral disc. Some embodiments may comprise providing access to the treatment area prior to inserting the expansible body therein. For example, an aperture may be provided in an annulus of an intervertebral disc to provide access to the nucleus thereof.

As shown in box 635, the method 600 further comprises maneuvering the expansible body to align the modified portion thereof with the identified tissue. For example, in one embodiment a surgeon may rotate a tool used to insert the expansible body into the treatment area to align a strengthened portion of an expansible body prosthesis with a tissue he or she has identified as having a particular characteristic (e.g., a tissue susceptible to further damage). At least the modified portion of the expansible body may comprise a radiopaque material the surgeon may monitor using imaging equipment while maneuvering the expansible body to at least partially align the modified portion with the identified tissue in the treatment area that may be damaged or susceptible to injury.

The method 600 shown in FIG. 11 finally comprises expanding the expansible body in the treatment area as shown in box 645. For example, in one embodiment, the expansible body may be at least partially filled with a biocompatible filler material while within the treatment area. The expansible body may then be sealed, and any equipment used to insert or fill it may then be removed from the treatment area.

The foregoing description of illustrative embodiments of the invention has been presented only for the purpose of illustration and description and is not intended to be exhaustive or to limit the invention to the precise forms disclosed. Numerous modifications and adaptations thereof will be apparent to those skilled in the art without departing from the spirit and scope of the present invention.

Furthermore, where methods and steps described above indicate certain events occurring in certain orders, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. Thus, the breadth and scope of the invention should not be limited by any of the above-described embodiments, but should be defined only in accordance with the following claims and their equivalents.

What is claimed is:

1. A device comprising:
an expansible body prosthesis configured to be deployed in a treatment area, the expansible body prosthesis comprising an aperture configured to receive a filler material, a first substantially impermeable portion having a first structural stiffness, and a second substantially impermeable portion contiguous to the first substantially impermeable portion and having a second structural stiffness greater than the first structural stiffness, wherein the second substantially impermeable portion is configured to be disposed adjacent a tissue in the treatment area at least when the expansible body prosthesis is in an expanded state.

2. The device of claim 1, wherein the first substantially impermeable portion comprises a first thickness, and the second substantially impermeable portion comprises a second thickness greater than the first thickness.

3. The device of claim 1, wherein the first substantially impermeable portion comprises a first body material, and the second substantially impermeable portion comprises a second body material different than the first body material.

4. The device of claim 1, further comprising a brace coupled to at least one of an interior surface and an exterior surface of the second substantially impermeable portion.

5. The device of claim 1, further comprising a coating material applied to at least one of an interior surface and an exterior surface of the second substantially impermeable portion.

6. The device of claim 1, wherein at least the second substantially impermeable portion comprises a cross-linkable material.

7. The device of claim 1, wherein the aperture is configured to be sealed, and wherein the second substantially impermeable portion comprises an analog of the aperture when the aperture is sealed.

8. The device of claim 1, wherein the expansible body prosthesis is configured to be deployed in a treatment area along a path established by a cannula.

9. The device of claim 1, wherein the expansible body prosthesis is configured to replace an intervertebral disc.

10. The device of claim 1, wherein the expansible body prosthesis is configured for placement inside an intervertebral disc.

11. The device of claim 1, wherein the expansible body prosthesis is configured to replace at least a portion of an intervertebral disc nucleus.

12. The device of claim 1, wherein the expansible body prosthesis further comprises a detachable coupling configured to detachably couple the expansible body prosthesis with an elongate member.

13. The device of claim 1, further comprising an annular buttress.

14. A device comprising:
an expansible body prosthesis configured to be deployed in a treatment area, the expansible body prosthesis comprising an aperture configured to receive a filler material, a first substantially impermeable portion, and a second substantially impermeable portion contiguous to the first substantially impermeable portion and configured to be disposed adjacent a tissue in the treatment area at least when the expansible body prosthesis is in an expanded state, wherein at least the second substantially impermeable portion of the expansible body prosthesis comprises a body material comprising a variable structural stiffness.

15. The device of claim 14, wherein the first substantially impermeable portion comprises a first thickness, and the second substantially impermeable portion comprises a second thickness greater than the first thickness.

16. The device of claim 14, wherein the first substantially impermeable portion comprises a first body material, and the second substantially impermeable portion comprises a second body material different than the first body material.

17. The device of claim 14, further comprising a brace coupled to at least one of an interior surface and an exterior surface of the second substantially impermeable portion.

18. The device of claim 14, further comprising a coating material applied to at least one of an interior surface and an exterior surface of the second substantially impermeable portion.

19. The device of claim 14, wherein at least the second substantially impermeable portion comprises a cross-linkable material.

20. The device of claim 14, wherein the aperture is configured to be sealed, and wherein the second substantially impermeable portion comprises an analog of the aperture when the aperture is sealed.

21. The device of claim 14, wherein the expansible body prosthesis is configured to be deployed in a treatment area along a path established by a cannula.

22. The device of claim 14, wherein the expansible body prosthesis is configured to replace an intervertebral disc.

23. The device of claim 14, wherein the expansible body prosthesis is configured for placement inside an intervertebral disc.

24. The device of claim 14, wherein the expansible body prosthesis is configured to replace at least a portion of an intervertebral disc nucleus.

25. The device of claim 14, wherein the expansible body prosthesis further comprises a detachable coupling configured to detachably couple the expansible body prosthesis with an elongate member.

26. The device of claim 14, further comprising an annular buttress.

27. A method comprising:
identifying a tissue in a treatment area;
modifying a structural stiffness of a portion of an expansible body prosthesis; and
inserting the expansible body prosthesis into the treatment area.

28. The method of claim 27, wherein identifying the tissue in the treatment area comprises identifying a characteristic of the tissue.

29. The method of claim 28, wherein modifying the structural stiffness of the portion of the expansible body prosthesis comprises modifying the structural stiffniess of the portion in relation to the characteristic of the tissue.

30. The method of claim 27, wherein identifying the characteristic of the tissue comprises identifying at least one of a damaged tissue, a weakened tissue, and a tissue susceptible to further damage.

31. The method of claim 27, wherein modifying the structural stiffness of the portion of the expansible body prosthesis comprises increasing the structural stiffness of the portion of the expansible body prosthesis.

32. The method of claim 27, wherein modifying the structural stiffness of the portion of the expansible body prosthesis comprises fabricating the portion of the expansible body prosthesis from at least one of a different thickness and a different body material than a second portion of the expansible body prosthesis.

33. The method of claim 27, wherein modifying the structural stiffness of the portion of the expansible body prosthesis comprises coupling a brace to at least one of an interior surface and an exterior surface of the portion.

34. The method of claim 27, wherein modifying the structural stiffness of the portion of the expansible body prosthesis comprises coating at least one of an interior surface and an exterior surface of the portion with a material configured to modify the structural stiffness of the portion.

35. The method of claim 27, wherein at least the portion of the expansible body prosthesis comprises a cross-linkable material, and wherein modifying the structural stiffness of the portion of the expansible body prosthesis comprises cross-linking the portion.

36. The method of claim 27, wherein modifying the structural stiffniess of the portion of the expansible body prosthesis comprises modifying the structural stiffness of the portion of the expansible body prosthesis prior to inserting the expansible body prosthesis into the treatment area.

37. The method of claim 27, wherein modifying the structural stiffness of the portion of the expansible body prosthesis comprises modifying the structural stiffness of the portion of the expansible body prosthesis subsequent to insertion of the expansible body prosthesis into the treatment area.

38. The method of claim 27, further comprising expanding the expansible body prosthesis in the treatment area.

39. The method of claim 27, further comprising maneuvering the expansible body prosthesis in the treatment area to align the portion of the expansible body prosthesis with the tissue.

40. The method of claim 27, further comprising detachably coupling the expansible body prosthesis an elongate member.

41. The method of claim 27, wherein the treatment area is located within an intervertebral disc.

42. The method of claim 41, wherein identifying the tissue in the treatment area comprises identifying a weakened area of an annulus of the intervertebral disc.

43. The method of claim 27, wherein the expansible body prosthesis is configured to replace an intervertebral disc.

44. The method of claim 27, wherein the expansible body prosthesis is configured for placement in an intervertebral disc.

45. The method of claim 27, wherein the expansible body prosthesis is configured to replace at least a portion of an intervertebral disc nucleus.

46. The method of claim 27, further comprising providing access to the treatment area.

47. The method of claim 46, and wherein providing access to the treatment area comprises providing access to a nucleus of an intervertebral disc through its annulus.

48. The method of claim 27, wherein inserting the expansible body prosthesis into the treatment area comprises inserting the expansible body prosthesis into the treatment area along a path established by a cannula.

49. A kit comprising:
a package configured to maintain its contents in a sterile condition; and
at least one sterilized expansible body prosthesis configured to be deployed in a treatment area, the sterilized expansible body prosthesis comprising an aperture configured to receive a filler material, a first substantially impermeable portion having a first structural stiffness, and a second substantially impermeable portion contiguous to the first substantially impermeable portion and having a second structural stiffness greater than the first structural stiffness, wherein the second substantially impermeable portion is configured to be disposed adjacent a tissue in the treatment area at least when the sterilized expansible body prosthesis is in an expanded state.

50. The kit of claim 49, further comprising:
a sterilized elongate member configured to at least temporarily couple with the aperture of the at least one expansible body prosthesis.

51. The kit of claim 49, further comprising:
a container at least partially filled with the filler material.

52. The kit of claim 49, further comprising:
a sterilized tool configured to at least one of cut and remove tissue.

53. The kit of claim 49, wherein the at least one sterilized expansible body prosthesis comprises a plurality of sterilized expansible bodies, wherein at least the second substantially impermeable portion of each sterilized expansible body prosthesis comprises a unique configuration.

54. The kit of claim 49, further comprising:
a sterilized brace configured to be coupled to at least one of an interior surface and an exterior surface of at least the second substantially impermeable portion of the at least one sterilized expansible body prosthesis.

55. The kit of claim 49, further comprising:
a coating material configured to be applied to at least one of an interior surface and an exterior surface of at least the second substantially impermeable portion of the at least one sterilized expansible body prosthesis.

56. The kit of claim 49, wherein at least the second substantially impermeable portion of the at least one sterilized expansible body prosthesis comprises a cross-linkable material; and further comprising:
a sterilized tool configured to cross-link at least a portion of the cross-linkable material.

57. The kit of claim 49, further comprising:
a sterilized access tool configured to provide at least one of access and navigation to the treatment area.

58. The kit of claim 49, further comprising:
a sterilized cannula configured to establish a path to the treatment area.

59. The kit of claim 49, wherein the expansible body prosthesis is configured to replace an intervertebral disc.

60. The kit of claim 49, wherein the expansible body prosthesis is configured for placement inside an intervertebral disc.

61. The kit of claim 49, wherein the expansible body prosthesis is configured to replace at least a portion of an intervertebral disc nucleus.

62. The kit of claim 49, further comprising an element configured to seal the aperture in the expansible body prosthesis when coupled to at least a portion of an edge of a body material of the expansible body prosthesis adjacent the aperture.

63. The kit of claim 49, further comprising a container at least partially filled with an adhesive configured to seal the aperture in the expansible body prosthesis when applied to least one of an interior surface and an exterior surface of a body material of the expansible body prosthesis.

64. A kit comprising:
a package configured to maintain its contents in a sterile condition; and
at least one sterilized expansible body prosthesis configured to be deployed in a treatment area, the at least one sterilized expansible body prosthesis comprising an aperture configured to receive a filler material, a first substantially impermeable portion, and a second substantially impermeable portion contiguous to the first substantially impermeable portion and configured to be disposed adjacent a tissue in the treatment area at least when the at least one sterilized expansible body prosthesis is in an expanded state, wherein at least the second substantially impermeable portion of the at least one sterilized expansible body prosthesis comprises a body material comprising a variable structural stiffness.

65. The kit of claim 64, further comprising:
a sterilized elongate member configured to at least temporarily couple with the aperture of the at least one sterilized expansible body prosthesis.

66. The kit of claim 64, further comprising:
a container at least partially filled with the filler material.

67. The kit of claim 64, further comprising:
a sterilized tool configured to at least one of cut and remove tissue.

68. The kit of claim 64, wherein the at least one sterilized expansible body prosthesis comprises a plurality of sterilized expansible bodies, wherein at least the second substantially impermeable portion of each sterilized expansible body prosthesis comprises a unique configuration.

69. The kit of claim 64, further comprising:
a sterilized brace configured to be coupled to at least one of an interior surface and an exterior surface of at least the second substantially impermeable portion of the at least one sterilized expansible body prosthesis.

70. The kit of claim 64, further comprising:
a coating material configured to be applied to at least one of an interior surface and an exterior surface of at least the second substantially impermeable portion of the at least one sterilized expansible body prosthesis.

71. The kit of claim 64, wherein the body material comprises a cross-linkable material; and further comprising:
a sterilized tool configured to cross-link at least a portion of the cross-linkable material.

72. The kit of claim 64, further comprising: a sterilized access tool configured to provide at least one of access and navigation to the treatment area.

73. The kit of claim 64, further comprising:
a sterilized cannula configured to establish a path to the treatment area.

74. The kit of claim 64, further comprising an element configured to seal the aperture in the expansible body prosthesis when coupled to at least a portion of an edge of a body material of the expansible body prosthesis adjacent the aperture.

75. The kit of claim 64, further comprising a container at least partially filled with an adhesive configured to seal the aperture in the expansible body prosthesis when applied to least one of an interior surface and an exterior surface of a body material of the expansible body prosthesis.

76. A method comprising:
detachably coupling an expansible body prosthesis comprising an aperture and configured to replace at least a portion of an intervertebral disc to a distal end of an elongate member;
inserting the expansible body prosthesis into a treatment area along a path established by a cannula;
at least partially expanding the expansible body prosthesis in the treatment area through the aperture; and
simultaneously sealing the aperture and detaching the elongate member from the expansible body prosthesis.

77. The method of claim 76, further comprising maneuvering the expansible body in the treatment area to detach the elongate member from the expansible body.

78. The method of claim 76, wherein the expansible body prosthesis comprises a frangible coupling configured to rupture upon a predetermined condition.

79. The method of claim 78, wherein the predetermined condition comprises an expansible body prosthesis interior pressure level reaching a predetermined threshold level.

80. The method of claim 78, wherein the predetermined condition comprises an expansible body prosthesis temperature level reaching a predetermined threshold level.

81. The method of claim 78, wherein the predetermined condition comprises an expansible body prosthesis interior fluid volume pressure level reaching a predetermined threshold level.
